(19) United States
(12) Patent Application Publication
(10) Pub. No.: US 2004/0072343 A1
(43) Pub. Date: Apr. 15, 2004

(54) Cell reprogramming

(76) Inventors: Paul John Verma, Scoresby (AU); Danielle Jacqueline Pralong, The Gap (AU); Peter David Rathjen, Blackwood (AU)

Correspondence Address: Sutherland Asbill & Brennan, 999 Peachtree Street NE, Atlanta, GA, 30309-3996, US

(21) Appl. No.: 10/416,361

(22) PCT Filed: Nov. 9, 2001
(86) PCT No.: PCT/AU01/01463

(30) Foreign Application Priority Data
Nov. 9, 2000 (AU) PR 1348
Dec. 19, 2000 (AU) PR 2162

(51) Int. Cl. C12N 5/08

(52) U.S. Cl. 435/366

(57) ABSTRACT

A method for reprogramming differentiated or partially differentiated cells to a less differentiated or dedifferentiated state, which method includes providing a karyoplast of a differentiated or partially differentiated cell; and a source of multinucleate, aneuploid, euploid or polyploid multipotent or pluripotent cells, or morula or early embryos, or derivatives thereof; and placing the karyoplast in intimate contact with the multipotent or pluripotent cells, or morula or early embryos or derivatives thereof under conditions sufficient to permit at least partial reprogramming of the cells.

The present invention relates to the reprogramming of differentiated or partially differentiated cells, for example blood (including bone marrow), skin, muscle, adipocyte or neural cells to a less differentiated state and to differentiated or partially differentiated cells subsequently derived therefrom. The present invention also relates to methods of identifying dedifferentiation of the dedifferentiated cells so formed.

Pluripotent cells can be isolated from the pre-implantation mouse embryo as embryonic stem (ES) cells. ES cells can be maintained indefinitely as a pluripotent cell population in vitro, and, when reintroduced into a host blastocyst, can contribute to all foetal and adult tissues of the mouse including the germ cells. ES cells, therefore, retain the ability to respond to all the signals that regulate normal mouse development, and potentially represent a powerful model system for the investigation of mechanisms underlying pluripotent cell biology and differentiation within the early embryo, as well as providing opportunities for genetic manipulation of the embryo and resultant commercial, medical and agricultural applications. Other pluripotent cells and cell lines, such as primordial germ cells (PGCs), embryonic carcinoma (EC) cells and early primitive ectoderm-like (EPL) cells, cells equivalent to the primitive ectoderm of post-implantation blastocysts (described in WO 99/53021), will share some or all of these properties and applications.

The differentiation of murine ES cells can be regulated in vitro by the cytokine leukaemia inhibitory factor (LIF) and other gp130 agonists, which promote self-renewal and inhibit differentiation of the stem cells. As outlined in WO 99/53021, to applicants differentiation of pluripotent cell in vitro can be directed in a uniform and precise manner to form an essentially homogeneous population of EPL cells, by cellular fibronectin and proline, or specific low molecular weight factors that include proline. EPL cells so formed can be maintained and proliferated in vitro. Unlike ES cell differentiation, EPL cell differentiation occurs in the absence of visceral endoderm and the inductive factors produced by visceral endoderm, allowing the production of cell populations essentially derived from a single germ layer. In summary there has been significant progress in developing methods for the precise control of pluripotent cell differentiation in vitro, resulting in the ability to form essentially homogeneous cell populations of ectodermal, mesodermal or endodermal origin.

International patent application WO97/32033 and U.S. Pat. No. 5,453,357 describe pluripotent cells including cells from species other than rodents, and primate pluripotent cells have been described in International patent applications WO98/43679 and WO96/22362 and in U.S. Pat. No. 5,843,780.

The availability of pluripotent cells and their differentiated progeny from a large number of animals, has widespread applications in animal health and farm management. Similarly human pluripotent cells offer novel approaches for the treatment of human diseases, where currently effective therapies are not available. EPL cells in particular, or cells obtained by directed differentiation of EPL cells, may be used in cell therapy and gene therapy, for the treatment of a number of diseases, including neurodegenerative diseases such as Parkinson's disease, and genetic diseases such as haemophilia, muscular dystrophy diseases and cystic fibrosis.

Although there have been major advances in regulating the differentiation of pluripotent cells in vitro, there is little information about how differentiated or partially differentiated cells can be induced to dedifferentiate to a pluripotent (e.g. ES) state.

Cell reprogramming is a process that alters or reverses the differentiation status of a partially differentiated cell or terminally differentiated cell. It includes reversion to a multipotent or pluripotent state, or transdifferentiation into a different cell type.

In the areas of cell therapy and gene therapy, reprogramming provides an approach for generating autologous pluripotent cells, which can then be differentiated along the desired differentiation pathway in a precisely controlled manner. Use of autologous cells in cell therapy offers a major advantage over non-autologous cells, which are likely to be subject to immunological rejection. Autologous cells are unlikely to elicit significant immunological responses (Munsie et al 2000).

Cells derived in this way offer potential cures for diseases such as Parkinson's disease and thalassemia, without problems caused by immunological rejection. Parkinson's disease is associated with degeneration of dopaminergic cells in the substantial nigra region of the brain. Dopaminergic cells may be derived in vitro from autologous pluripotent cells (generated by reprogramming of somatic cells isolated from the patient), and implanted into the substantia nigra to replace the dysfunctional dopaminergic cells.

Reprogramming of nuclei from differentiated cells occurs during nuclear transfer, when karyoplasts are transferred to enucleated ova.

The term “karyoplast”, as used herein, may be defined as a nucleus of a differentiated or partially differentiated cell surrounded by a plasma membrane. It includes an intact cell, and nuclei surrounded by a pool of cytoplasm.

Nuclear transfer has resulted in the production of viable embryos and offspring genetically identical to the donor nuclei. Nuclear transfer was first performed with amphibians (Gurdon, 1974) and has more recently been established in mammals. For example sheep clones were produced using nuclei from embryonic blastomeres as karyoplasts (Willadsen, 1986), and similarly cloning of cow (Prather et al, 1987) and rabbit (Stice & RobI, 1988) was also achieved using early embryonic cells as the source of donor nuclei. Further development of nuclear transfer technology has resulted in the production of live sheep derived from karyoplasts from foetal cells (Campbell et al, 1996) and adult cells (Wilmut et al, 1997). Animal cloning offers, for the first time, an approach for the precise genetic manipulation of livestock, with consequent benefits in animal husbandry, and in human xenotransplantation.

Most significantly, nuclear transfer has shown that oocyte cytoplasm provides an environment for the reprogramming of partially and terminally differentiated cells into cells with the differentiation capacity of early embryonic cells.

Several factors may influence the reprogramming of karyoplasts, including:

Cell cycle synchronisation of karyoplast and recipient cytoplast (Campbell et al, 1996).

Timing of transcriptional onset in the recipient cell (Smith and Wilmut, 1989).

Methylation status (Kikyo and Wolffe, 2000).

The use of enucleated oocytes to produce nuclear transfer embryos for the derivation of cells equivalent to early embryonic cells is central to the technology currently available for reprogramming and the isolation of autologous pluripotent cells. This approach for the derivation of autologous human pluripotent cells has been termed “therapeutic cloning”. Ethical barriers related to the use of human early embryos and human cloning are major obstacles to the further development of this technology, and its widespread clinical use. There are also ethical and practical difficulties associated with routine supply of human oocytes for use as cytoplasts.

Animal oocytes (eg bovine oocytes) have been proposed as an alternative source of recipient cells for human karyoplasts, for the development of autologous pluripotent cells. However this approach has introduced separate strong ethical objections. Furthermore the production, developmental capacity and long term viability of embryonic cells derived from karyoplasts and cytoplasts from different species has not been established.

More recently there have been examples of transdifferentiation in vivo, where adult stem cells appear to have a less restricted differentiation capacity than previously believed. For example, adult neural stem cells contributed to haematopoietic lineages when injected into the circulation (Bjomson et al, 1999), and conversely adult bone marrow stem cells injected into the circulation of irradiated mice, contributed to glia in various regions of the brain (Eglitis & Mezey, 1997), to new skeletal muscle (Ferrari et al, 1998), and to hepatic precursor cells (Petersen et al, 1999).

In principle the apparent plasticity of adult stem cells, and their ability to differentiate into cells of a different germ lineage could be applied in autologous cell therapy and gene therapy. However the mechanisms responsible for transdifferentiation are not understood, and technology to regulate transdifferentiation has not been developed, currently limiting its utility in the clinic.

The ability of pluripotent cells to reprogram somatic cell nuclei is suggested in experiments involving pluripotent cell-somatic cell heterokaryons, where pluripotent characteristics are retained (Tada et al (1997, Matveeva et al (1998), WO 00/49137). However it remains possible that different pluripotent cell types vary in their capacity for reprogramming. For example, there may be variation in the ability of different pluripotent cell types to reset epigenotype characteristics of differentiated cells, such as methylation patterns, to those of pluripotent cells.

Further, the concept of using cytoplasts derived from pluripotent cells to reprogram somatic cell nuclei is disclosed in WO 00/49137. This disclosure, however, does not address the technical difficulties associated with pluripotent cytoplasts, such as pluripotent cytoplast preparation, the introduction of somatic cell nuclei into pluripotent cytoplasts or other pluripotent environments, and the reprogramming of somatic cell nuclei by pluripotent cytoplasts or other pluripotent environments.

It is an object of the present invention to overcome, or at least alleviate, one or more of the difficulties or deficiencies associated with the prior art.

Applicant has surprisingly found that differentiated or partially differentiated cells, e.g. blood (including bone marrow), skin, muscle, adipocyte or neural cells, may be induced to dedifferentiate via intimate contact, e.g. fusion with multinucleate or polyploid multipotent or pluripotent cells, early embryos or derivatives thereof.

Accordingly, in a first aspect of the present invention there is provided a method for reprogramming differentiated or partially differentiated cells to a less differentiated or dedifferentiated state, which method includes

providing

a karyoplast of a differentiated or partially differentiated cell; and

a source of multinucleate, aneuploid, euploid or polyploid multipotent or pluripotent cells, or morula or early embryos, or derivatives thereof; and

placing the karyoplast in intimate contact with the multipotent or pluripotent cells, or morula or early embryos or derivatives thereof under conditions sufficient to permit at least partial reprogramming of the cells.

The karyoplasts of differentiated or partially differentiated cells may be of any suitable type. The karyoplasts may include an intact cell or a nucleus surrounded by a pool of cytoplasm.

Autologous cells may be used. Any differentiated or partially differentiated cell that can be obtained, e.g. by biopsy, from a patient may be used. Blood (including bone marrow), skin, muscle, adipocyte, neural or like cells are preferred. More preferably blood cells, e.g. blood karyoplasts, may be used.

As used herein, the term “pluripotent” refers to cells that can contribute substantially to all tissues of the developing embryo. “Multipotent” or “partially differentiated” refers to partially differentiated cells that are able to differentiate further into one or more than one terminally differentiated cell type. Such cells include, but are not limited to, haematopoietic stem cells and neural stem cells.

The multipotent or pluripotent cells utilised for reprogramming may be derived from cells selected from the group consisting of embryonic stem cells (ES cells), early primitive ectoderm-like cells (EPL cells), primordial germ cells (PG cells) and embryonic carcinoma cells (EC cells); or derivatives thereof or mixtures thereof.

ES cells retain pluripotence indefinitely and display the properties of stem cells, including competency to differentiate into all cell types, and the ability for indefinite self-renewal. Early primitive ectoderm-like (EPL) cells are also pluripotent stem cells. They differ in many properties to ES cells, but have the capacity to revert to ES cells in vitro. They can be derived from ES cells or other types of pluripotent cells, and are the in vitro equivalent of primitive ectoderm cells of post-implantation embryos. As such, EPL cells can also be established in vitro from cells isolate from the primitive ectoderm of post-implantation embryos. The properties of EPL cells, factors required for their maintenance and proliferation in vitro, and their ability to differentiate uniformly in vitro to form essentially homogeneous populations of partially differentiated and differentiated cell types are described fully in WO99/53021, to applicants, the entire disclosure of which is incorporated herein by reference. Cells of the primordial gonad, primordial germ cells (PGCs), also retain pluripotency during embryonic development, and can be isolated and cultured in vitro. Embryonic carcinoma (EC) cells may also be pluripotent.

In addition pluripotent cells may be derived by dedifferentiation (e.g. by reverting differentiated cells to a pluripotent state), or by application of nuclear transfer techniques, (e.g. when the nucleus of a differentiated or partially differentiated cell is transferred into an oocyte or early embryonic cell).

The multipotent or pluripotent cell source may take the form of embryoid bodies (EBs) derived from ES or EPL cells in vitro. Differentiated or partially differentiated cells may be incorporated within the EB or aggregated with cells of the EB for time sufficient for at least partial reprogramming to occur.

The multipotent or pluripotent cell source is preferably a multinucleate or polyploid pluripotent cell.

Preferably, the multinucleate or polyploid pluripotent cell is a polyploid ES or EPL cell; and

the differentiated or partially differentiated cells are blood or neural cells.

The pluripotent cells may be from any vertebrate including murine, human, bovine, ovine, porcine, caprine, equine and chicken. The cells may be isolated by any method known to the skilled addressee.

Desirably, cytoplasts derived from ES or EPL cells may be used.

Alternatively where embryonic cells are utilised as hosts for reprogramming, embryos, preferably early embryos, for example at the morula stage, are preferred. Derivatives thereof or call extracts thereof may be used. Embryos from cross-species, e.g. primate, mouse, bovlne, ovine, etc. embryos may be used to reprogram mammalian cells.

The cell contact step according to this aspect of the present invention may take any suitable form. Where multipotent or pluripotent cells are used, the cell contact step may preferably include

subjecting the cells to a cell fusion step.

Fusion of cells, for example in suspension, may be achieved by electrical pulse or by exposure to polyethylene glycol (PEG). Alternatively cells in monolayer culture, where cell-cell contact occurs, may be fused by exposure to PEG (Roos, 1991). A combination of electrofusion and PEG treatment may also be used.

In an optional aspect of the present invention, where the cells achieve only partial reprogramming at first instance, the reprogramming step may be repeated one or more times. Accordingly, in this aspect, when the cells are only partially reprogrammed, the method according to the present invention may further include

isolating the partially reprogrammed cells; and

repeating the contact step until reprogramming is complete

In a preferred aspect of the present invention, there is provided a method for reprogramming differentiated or partially differentiated cells to a less differentiated state, or de-differentiated state, which method includes

providing

a karyoplast of a differentiated or partially differentiated cell;

a source of mammalian morula or early embryos or derivatives thereof;

placing the karyoplast cells in intimate contact with morula or early embryos or derivatives thereof for a period sufficient to permit at least partial reprogramming of the cells.

Preferably a mammalian karyoplast is placed in the perivitelline space of a mammalian early embryo.

In a preferred aspect, the method may further include

separating the reprogrammed cells from other cells.

The separation step may include

providing a source of differentiated or partially differentiated cells genetically modified to include a selectable marker; and

selecting the reprogrammed cells utilising the selectable marker. The selectable marker may include a marker which enables separation of the reprogrammed cells from the embryonic cells.

Preferably the selectable marker is a marker expressed in pluripotent or multipotent cells.

Preferably the selectable marker contains a gene encoding Green Fluorescent Protein (GFP) linked to an Oct4 promoter, and preferably fluorescent activated cell sorting (FACS) is used to separate reprogrammed cells.

More preferably, when the cells are only partially reprogrammed, at first instance, the method may further include

isolating the partially reprogrammed cells; and

repeating the contact step until reprogramming is complete.

In an alternative preferred aspect of the present invention, there is provided a method for reprogramming differentiated or partially differentiated cells to a less differentiated, or dedifferential state, by formation of a cell hybrid, which method includes

providing:

a source of karyoplasts of differentiated or partially differentiated cells;

a source of multipotent or pluripotent cells, morula or early embryos or derivatives thereof;

placing the karyoplasts in intimate contact with the multipotent or pluripotent cells, or morula or early embryo cells or derivatives thereof;

subjecting the cells to a cell fusion step to form a cell hybrid;

subsequently removing from said reprogrammed cells, the multipotent or pluripotent cell chromosomes; and

culturing the reprogrammed cells to permit proliferation thereof.

Preferably the differentiated cell or partially differentiated cell source is a somatic cell. Preferably the multipotent or pluripotent cells are multinucleate, aneuploid, euploid or polyploid multipotent or pluripotent cells.

The cell fusion step may include subjecting the cells to an electrical pulse or exposure to polyethylene glycol (PEG) or a combination thereof.

The cell fusion step may be such that the nuclei of the cell components remain separated; and

subsequently removing from said reprogrammed cells, the multipotent or pluripotent cell nuclei.

More preferably during the cell fusion step the somatic cell nucleus and the nucleus of multipotent or pluripotent cells are maintained as separate nuclei by maintaining the cells at low temperature, by utilising a cell cycle arrester, such as aphidocolin, or a cytoskeletal inhibitor (e.g. cytochalasin B, cytochalasin D) or combinations thereof.

In the enucleation step, the multipotent or pluripotent cell nucleus may be removed by differential centrifugation, or the reprogrammed cells may exhibit spontaneous removal of the multipotent or pluripotent cell nucleus.

In a further aspect of the present invention, there is provided a method for preparing a multinucleate, aneuploid, euploid or polyploid multipotent or pluripotent cell, which method includes

providing a source of pluripotent and/or multipotent cells; and

subjecting the cells to a fusion step to produce a multinucleate, aneuploid, euploid or polyploid cell.

Preferably the pluripotent cells used are embryonic stem (ES) cells or early primitive ectoderm (EPL) cells.

The cell fusion step according to this aspect of the present invention leads to the production of large, multinucleate, aneuploid, euploid or polyploid cells that contain an increased pool of cytoplasm. Fused cells may contain two or more diploid (unfused) nuclei, or polyploid (at least 4N) chromosomes.

The cell fusion step may take any suitable form. Fusion of cells in suspension may be achieved by electrical pulse or by exposure to PEG. Alternatively cells in monolayer culture, where cell-cell contact occurs, may be fused by exposure to PEG (Roos, 1991). A combination of electrofusion and PEG treatment may also be used. In addition fused cells may be cultured to produce a polyploid (4N or greater) cell line which may be used for enucleation.

The multinucleate, aneuploid, euploid or polyploid pluripotent cells according to the present invention are of particular advantage in intercellular nuclear transfer (the transfer of a differentiated or partially differentiated karyoplast into a pluripotent or multipotent cell or cytoplast).

In a still further aspect of the present invention, there is provided multinucleate, aneuploid, euploid or polyploid multipotent or pluripotent cells derived from fusion of two or more diploid cells.

The multipotent or pluripotent cell source according to the present invention may accordingly preferably include a source of multi-nucleate or polyploid pluripotent cells.

The multinucleate, aneuploid, euploid or polyploid cells may be expanded to generate a cell line. The cell line is preferably stable in culture in vitro.

Accordingly, in a further aspect of the present invention there is provided a cell line formed from the multinucleate, aneuploid, euploid or polyploid pluripotent or multipotent cells described above.

Significantly applicants have surprisingly established that pre-fusion of pluripotent cells provide larger cells with sufficient cytoplasm for intercellular nuclear transfer for reprogramming of somatic cell nuclei. Application of this concept to pluripotent cells, may overcome one or more of the technical difficulties imposed by the unusually large nucleus/cytoplasm ratio for pluripotent cells., which are usually diploid.

Larger cytoplasts offer several advantages in intercellular nuclear transfer.

1. Enucleation efficiency using any enucleation technique, including micromanipulation, density gradient enucleation or centrifugal enucleation is increased and/or enucleation is facilitated.

2. Efficiency of cell reconstruction by combining a karyoplast with a pluripotent or multipotent cytoplast is improved, since it increases karyoplast/cytoplast contact.

3. A larger cytoplasmic pool is available to induce reprogramming.

Accordingly, in a further aspect of the present invention, there is provided a method for the production of a reprogrammed cell which method includes:

providing

a source of cytoplasm from a multinucleate, aneuploid, euploid or polypoid multipotent or pluripotent cell;

a karyoplast derived from a partially or terminally differentiated cell differing from the cytoplasm source;

a suitable culture medium

combining the cytoplasm source and karyoplast to form a reconstructed cell; and

maintaining the reconstructed cell in the culture medium for a time sufficient to permit at least partial reprogramming of the karyoplast in the combined cell.

Preferably the cell reconstruction step includes fusion of the karyoplast and cytoplast so that the cytoplast content interacts with the karyoplast and induces at least partial reprogramming of the nuclear material.

The source of cytoplasm may be cytoplasm removed from multinucleate, aneuploid, euploid or polyploid cells or may be cytoplasm derived by at least partial enucleation of said cells. Preferably the source of cytoplasm is an enucleated pluripotent fused cell.

The source of cytoplasm may be cytoplasm removed from ES or EPL cells or may be cytoplasm derived by at least partial enucleation of said cells. Preferably the source of cytoplasm is enucleated multinucleate, aneuploid, euploid or polyploid ES or EPL cells.

In a preferred form, when the cells are only partially reprogrammed, the method according to the present invention further includes

isolating the partially reprogrammed cells; and

repeating the reconstruction and maintenance steps until reprogramming is complete.

In a further aspect of the present invention, there is provided a method for preparing a cytoplast, which method includes

providing a multinucleate, aneuploid, euploid or polyploid multipotent or pluripotent cell;

subjecting the multipotent or pluripotent cell to an enucleation step; and

harvesting the cytoplast so formed.

Cytoplasts prepared from fused pluripotent cells may have a greater content of factors necessary for reprogramming of somatic nuclei, and are more readily manipulated.

Enucleation of fused cells may be performed in the presence of cytochalasin B, a cytoskeletal disrupting agent. It may be conducted by micromanipulation, using a fine pipette to aspirate and pinch off droplets of cytoplasm from fused cells. However, this approach is tedious and time consuming, and may not be generally useful for the production of large numbers of cytoplasts.

Other approaches, such as density gradient centrifugation (Poste, 1972) and centrifugal enucleation may be used. These techniques are rapid and allow the preparation of cytoplasts in bulk. Density gradients such as discontinuous Percoll density gradients separate organelles on the basis of their different density. Nuclei, the denser organelle, are extruded and separate from cytoplasts, which float at a lighter density.

Centrifugal enucleation is conducted with cells adhered to an inverted disc, such as a gelatinised plastic disc. Centrifugation leads to extrusion of the nucleus into the medium, while cytoplasts remain adhered to the disc (Prescott et al., 1971).

Accordingly, in a further aspect of the present invention there is provided a cytoplast derived from a multinucleate, aneuploid, euploid of polyploid multipotent or pluripotent cell as described above.

Karyoplasts are nuclei that are surrounded by a plasma membrane, and are used in nuclear transfer, or intercellular nuclear transfer. As stated above, the term “karyoplast as used herein includes an intact cell. Karyoplasts can be fused with cytoplasts, resulting in reprogramming of the karyoplast nucleus.

Accordingly, in a still further aspect of the present invention, there is provided a method for preparing a karyoplast which method includes

providing a partially or terminally differentiated cell;

subjecting the partially or terminally differentiated cell to an enucleation step; and

harvesting the karyoplast so formed.

For the preferred derivation of autologous cells to be used in cell therapy, karyoplasts are obtained from partially differentiated or differentiated somatic cells, e.g. cells biopsied from the patient.

The source of karyoplasts may be somatic cells, e.g. wherein the somatic cell is a blood (including bone marrow), skin, muscle, adipocyte or neural cell.

Karyoplasts may be obtained from differentiated or partially differentiated cells by micromanipulation density gradient centrifugation or centrifugal enucleation in the presence of the cytoskeletal disrupting agent, cytochalasin B. These procedures allow isolation of karyoplasts with minimal cytoplasmic component. Alternatively entire cells may be used as karyoplasts.

The cell combination or cell reconstruction step may include fusion of karyoplasts and cytoplasts, so that the cytoplast content interacts with the karyoplast, and induces reprogramming of the nuclear material. The reprogrammed or dedifferentiated cell may then be permitted to differentiate in a controlled manner, along the desired differentiation pathway to form partially differentiated or terminally differentiated cells, e.g. with Cell Therapy applications, as discussed below.

The cytoplasts and karyoplasts combination step may include fusion utilising electrofusion, or PEG-mediated fusion, or a combination thereof, or any other known method used to fuse cells such as Sendai virus etc. An agglutination treatment such as phytohaemagglutinin, to increase karyoplast-cytoplast contact may preferably be used to improve the efficiency of reconstruction.

The reprogramming step is such that the karyoplasts or nuclei from partially or terminally differentiated cells are dedifferentiated.

Alternatively, where embryos are used, the cell contact step may include introducing the karyoplast from the differentiated or partially differentiated cells into an embryo.

Introduction of the karyoplasts may be by way of micro-injection. The injected cells are maintained in contact with the embryo, for example in a suitable culture medium, for a period sufficient to permit reprogramming or injected cells to be complete. The cells may be maintained for a period of approximately 24 to 96 hours, preferably approximately 72 hours.

In a further aspect of the present invention, there is provided a reprogrammed pluripotent or multipotent cell., produced by the methods according to the present invention.

In one form of this aspect of the present invention, the cells may be maintained in a suitable culture medium in the presence of a factor or factors that promote maintenance of a multipotent or pluripotent state. For pluripotent cells, factors may include a gp 130 agonist such as the cytokine leukaemia inhibitory factor (LIF) preferably at a concentration of greater than approximately 100 units/ml and more preferably greater than approximately 1000 units/ml. Oncostatin M, CNTF, CT1 or IL6 with the soluble IL6 receptor, and IL11 and other gp130 agonists at equivalent levels may also be used.

Preferably the cells may be cultured in the presence of a suitable factor or factors under conditions suitable for their proliferation and maintenance in vitro. This includes the use of serum including fetal calf serum and bovine serum or the medium may be serum-free. Other growth enhancing components such as insulin, transferrin and sodium selenite may be added to improve growth of the cells. As would be readily apparent to a person skilled in the art, the growth enhancing components will be dependent upon the cell types cultured, other growth factors present, attachment factors and amounts of serum present.

The cells may be cultured for a time sufficient to establish the cells in culture. By this we mean a time when the cells equilibrate in the culture medium. Preferably the cells are cultured for approximately 2 to 14, preferably 3 to 10, days.

The cell culture medium may be any cell culture medium appropriate to sustain the cells employed. The culture medium is preferably DMEM containing high glucose, supplemented with 10% FCS, 1 mM L-glutamine, 0.1 mM βME, 37° C., 5% CO₂.

The reprogramming of differentiated or partially differentiated cells to a less differentiated, e.g. pluripotent state may be assessed by expression of marker genes (RNA transcripts, proteins and cell surface markers), cell morphology, cytokine responsiveness and/or by subsequent differentiation in vitro or in vivo.

In a preferred aspect of the present invention, applicants have developed a method for identifying, which and/or when, cells are reprogrammed in a cell mixture which contains reprogrammed and unmodified cells.

Accordingly, the present invention further provides a method for identifying dedifferentiation of differentiated or partially differentiated cells, which method includes

providing a cell mixture including reprogrammed or partially reprogrammed cells, produced according to any one of claims 1 to 37 the reprogrammed cells being optionally modified to include a pluripotent marker; and

screening the cell mixture for the presence or absence of pluripotent activity.

Preferably, when the reprogrammed cells are modified to include a pluripotent marker,

subsequently screening the cell mixture for the presence or absence of pluripotent marker activity.

More preferably the reprogramming step includes

placing karyoplasts of differentiated or partially differentiated cells in intimate contact with the multipotent or pluripotent cells or morula or early embryos under conditions sufficient to permit at least partial reprogramming of the cells.

The multipotent or pluripotent cells are preferably multinucleate, aneuploid, euploid or polyploid multipotent or pluripotent cells, as described above.

More preferably the differentiated or partially differentiated cells are neurectoderm cells, as described below.

The cell construct according to this aspect of the present invention may include an ES or EPL cell line genetically modified to express a marker gene.

For example, an ES cell line may be transfected with an Oct4-TK-GFP construct according to the present invention, and used to derive the differentiated or partially differentiated karyoplasts (e.g. neurectoderm cells) according to WO 99/53021. Differentiated or partially differentiated karyoplasts that do not express the Oct4-TK-GFP construct, and do not fluoresce, may be fused with cytoplasts produced from tetraploid ES cells.. Initially no fluorescence is observed. After a sufficient period has elapsed, fluorescence, indicative of pluripotency, will begin to emerge.

Marker genes which may be used to identify cells reprogrammed to a pluripotent state include markers selected from the group consisting of Rex1, Fgf5, Oct4, alkaline phosphatase, uvomorulin, AFP, H19, Evx1, brachyury, and novel marker genes, identified by the inventors, such as L17, Psc1 and K7. Marker genes down regulated during transition from ES cells to EPL cells include Rex1, L17and Psc1. Fgf5 and K7are up regulated during this transition. Pluripotent cell markers Oct4, Alkaline phosphatase and uvomorulin are expressed by both ES cells and EPL cells in similar levels. Other genes that are expressed in partially differentiated or differentiated embryonic or extraembryonic lineages such as AFP, H19, Evx1 and brachyury are not expressed in any ES or EPL cells. The pluripotent cell marker Oct 4 is preferred.

The cells may be modified to generate a visible marker, such as a fluorescent marker. The cells may include an ES cell line, which expresses GFP under the control of elements of the Oct 4 promoter.

The construct used to transfect ES cells may be an Oct 4-TK-GFP construct The construct may be an Oct 4-TK-GFP-IRES-Puro construct. The construct is antibiotic (Puromycin) resistant.

The ES or EPL cells may be maintained in a pluripotent state by culture in the presence of the biologically active factor or components thereof or the conditioned medium or the extracellular matrix optionally plus low molecular weight component, in the presence or absence of additional factors that maintain pluripotency (e.g. a gp130 agonist), until further differentiation induced by factors, conditions or procedures, is initiated.

Modification of the genes of the reprogrammed cells may be conducted by any means known to the skilled person which includes introducing extraneous DNA, removing DNA or causing mutations within the DNA of these cells. Modification of the genes includes any changes to the genetic make-up of the cell thereby resulting in a cell genetically different to the original cell.

In a preferred aspect of the present invention, there is provided a method for the production of differentiated or partially differentiated cells which method includes:

providing

a source of reprogrammed pluripotent or multipotent cells, preferably autologous cells, as described above; and

a suitable medium; and

culturing the reprogrammed cells, optionally in the presence of a selected growth factor, for a period sufficient to permit partial or terminal differentiation.

Suitable differentiation methods may be as described in WO 99/53021, WO 01/51610 and WO 01/51611 to applicants, the entire disclosure are incorporated herein by reference.

Accordingly, in a still further aspect of the present invention there is provided a chimaeric or transgenic animal, including animals derived by nuclear transfer, produced using a cell or produced according to the present invention.

The pluripotent and multipotent cells produced according to the present invention have wide ranging applications.

For example they have applications in livestock management, involving the precise genetic manipulation of animals for economic or health purposes.

Pluripotent and multipotent cells obtained according to the present invention, and preferably their differentiated progeny obtained by programmed or directed differentiation, have applications of particular significance in cell therapy and gene therapy, for the treatment of human disease.

The preferred mode of use is to use autologously-derived ES or EPL cells and their progeny, thereby avoiding immunological rejection of implanted cells.

Cells programmed to form ectodermal lineages may be used for cell therapy procedures including but not restricted to neuronal and dermal cell therapy procedures. In particular they can be used to treat and cure neurodegenerative disorders such as Parkinson's disease, Huntington's disease, lysosomal storage diseases, multiple sclerosis, memory and behavioural disorders, Alzheimer's disease and macular degeneration, and other pathological conditions including stroke and spinal chord injury. For example reprogrammed cells may be used to derive genetically modified or unmodified neurectoderm cells or their differentiated or partially differentiated progeny, which in turn may be used to replace or assist the normal function of diseased or damaged tissue. In the case of Parkinson's disease, the dopaminergic cells of the substantia nigra are progressively lost. The dopaminergic cells in Parkinson's patients may be replaced by implantation of neurectoderm or partially differentiated or differentiated neuronal cells.

Reprogrammed cells may also be used for to derive neural crest cells and their differentiated or partially differentiated progeny for the treatment of spinal cord disorders, and Schwann cells for the treatment of multiple sclerosis. Neural crest cells also retain the capacity to form non-neural cells, including cartilage and connective tissue of the head and neck, and are potentially useful in providing tissue for craniofacial reconstruction.

In further applications, reprogrammed may can be used to derive precursors of adult skin, hairs, lens and cornea of the eye, including surface ectoderm and its derivatives for transplantation therapy. A number of corneal disorders may be treated by corneal transplant, including corneal clouding, degeneration following cataract surgery, keratoconus, bullous keratopathy and chemical burns. Currently corneas for transplant are sourced from deceased donors. Similarly skin grafts are used to treat a number of conditions, most notably burns.

Surface ectoderm derived in vitro from reprogrammed cells may be further differentiated into a number of surface tissues including corneal epithelia, skin and lens, providing an alternative source in potentially unlimited amounts of those tissues for transplant.

Cells programmed for mesodermal lineages may be used for cell therapy procedures including but not restricted to bone marrow and muscle cell therapies; for example, for the treatment of cancer, for bone marrow rescue and regeneration of cardiac muscle following heart attack.

Cells derived according to present invention also have widespread uses in human gene therapy. Such gene therapy may preferably be conducted using autologously-derived ES or EPL cells or their differentiated progeny. Reprogrammed cells or their differentiated and partially differentiated products may be genetically modified to treat diseases such as, but not restricted to haemophilia, diabetes type 1, Ducheynne's and other muscular dystrophies, Gauche's disease and other mucopolysaccharide diseases, and cystic fibrosis. Genetically modified reprogrammed cells may also be used in the treatment of skin, dye or hair diseases. Reprogrammed cells may also be genetically modified so that they provide functional biological molecules such as cytokines or lymphokines (e.g. interleukin-2). The genetically modified cells may be implanted, thus allowing appropriate delivery of therapeutically active molecules for the treatment of cancers and other diseases.

Unmodified or genetically modified ES or EPL cells and preferably their differentiated progeny obtained by programmed or directed dedifferentiation may be used to generate cells and tissues and components of organs for transplant.

In a preferred embodiment of this further aspect of the present invention, there is provided a method for the production of a chimeric animal, which method includes

providing

a reprogrammed multipotent or pluripotent cell produced according to the present invention, and

a pregastrulation embryo;

introducing the multipotent or pluripotent cell into the pregastrulation embryo; and

monitoring chimera forming ability.

The present invention will now be more fully described with reference to the accompanying examples and drawings. It should be understood, however, that the description following is illustrative only and should not be taken in any way as a restriction on the generality of the invention described above.

In the figures:

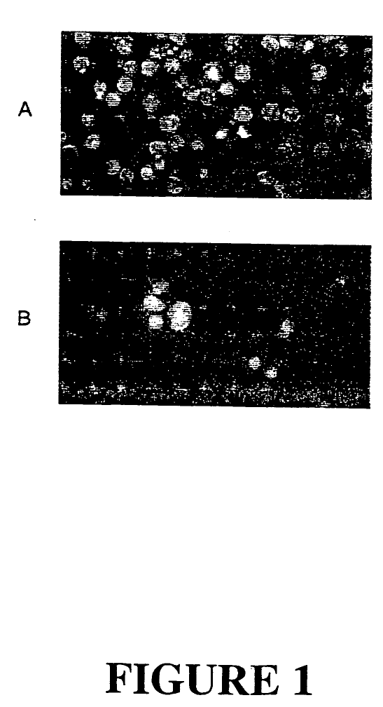

FIG. 1: ES cell fusion

A Unfused normal diploid ES cells.

B Larger fused ES cells. Fusion rates of 10% to 30% have been observed.

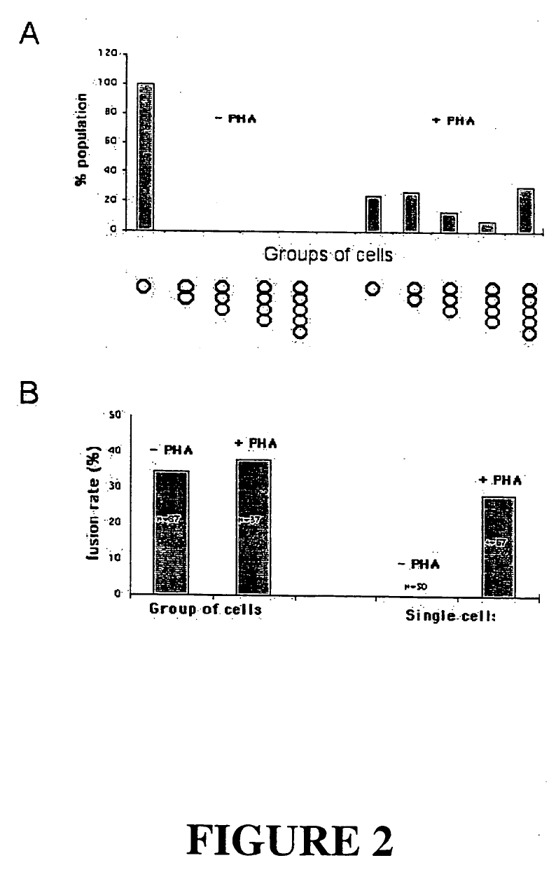

FIG. 2: Phytohaemagglutinin promotes cell agglutination and cell fusion

A Effect of PHA on ESD3 cell agglutination.

B Effect of PHA on ESD3 cell fusion.

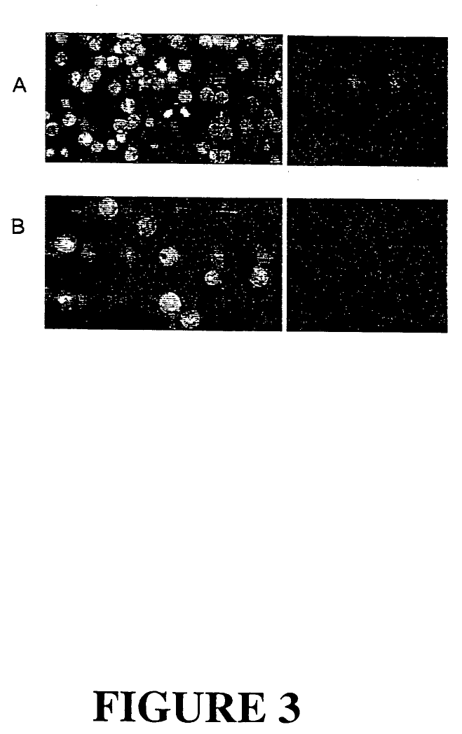

FIG. 3: Fused ES ell line

A Unfused normal diploid ES cells.

B Double antibiotic selected 4N ES cell line. Note larger cytoplasm content compared to A.

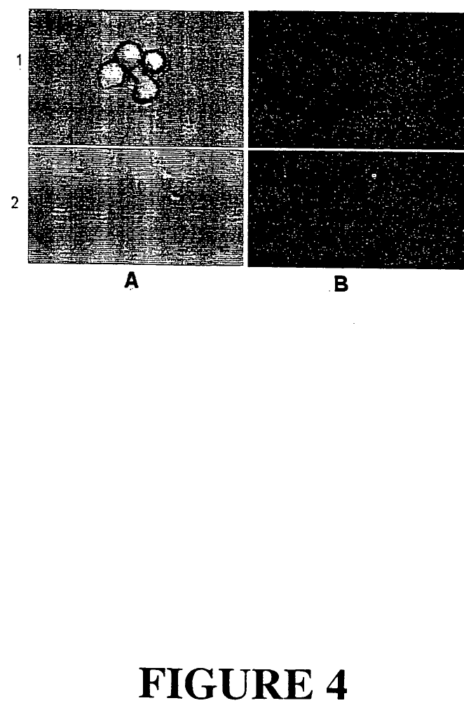

FIG. 4: Cytoplasts and karyoplasts prepared by density gradient centrifugation

1A Cytoplasts viewed under transmitted light/brightfield microscopy

1B The same field viewed under UV2A fluorescence. Note lack of staining with Hoechst 33342.

2A Karyoplasts viewed under transmitted light field microscopy.

2B The same field viewed under UV2A fluorescence. Note intense nuclear staining with Hoechst 33342.

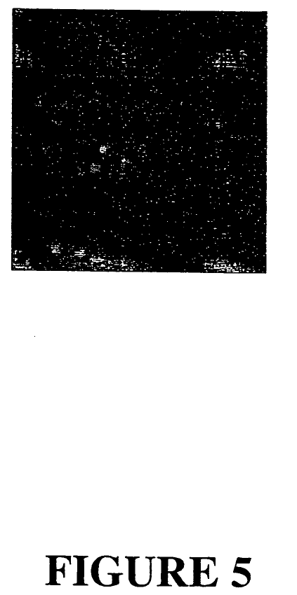

FIG. 5: Enriched cytoplast preparation

Cytoplasts produced by Percoll gradient centrifugation were enriched by a second round of density gradient centrifugation. This resulted in a preparation containing up to 80% cytoplasts.

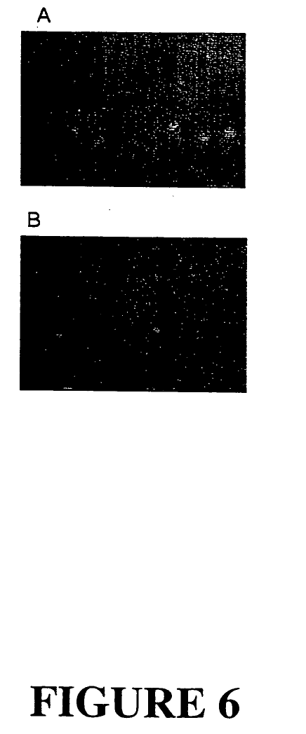

FIG. 6: Cytoplasts prepared by centrifugal enucleation

Top panel:

Cells and cytoplasts attached to discs were stained with Hoechst 33342 following enucleation. Structures were viewed using a combination of bright field and UV2A fluorescence microscopy. Nucleated cells are identified by the presence of blue fluorescence. Cytoplasts are identified as structures lacking a nucleus.

Bottom panel:

The same disc viewed using only UV2A fluorescence microscopy to confirm the nucleated/denucleated status of cells and cytoplasts.

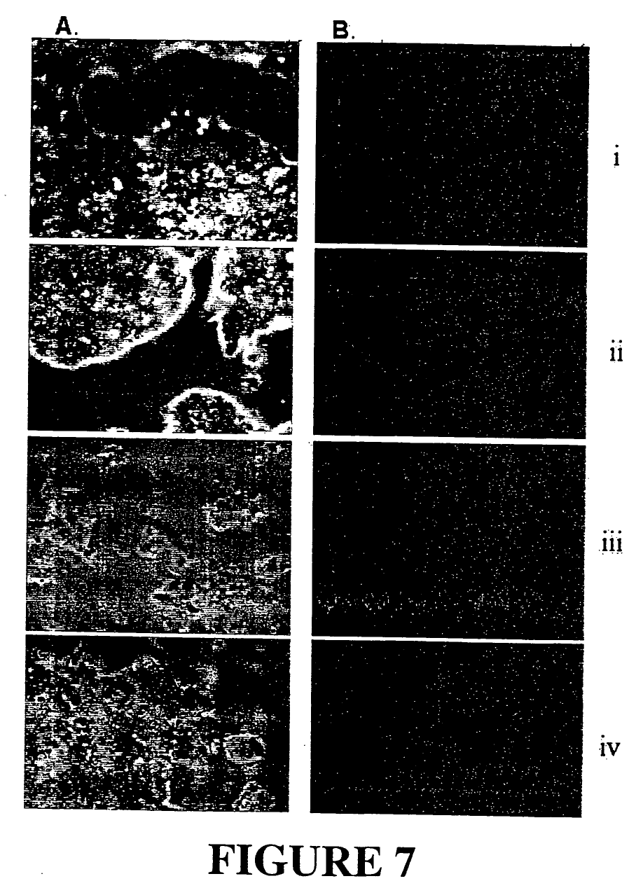

FIG. 7: Fusion of ES cell karyoplasts with intact ES cells

Karyoplasts were prepared from ES cells expressing cytoplasmic GFP and puromycin resistance. Karyoplasts were essentially devoid of cytoplasm, and were fused to intact ES cells. Fused cells were viewed for cytoplasmic GFP expression.

Column A Phase contrast microscopy.

Column B Epifluorescence microscopy.

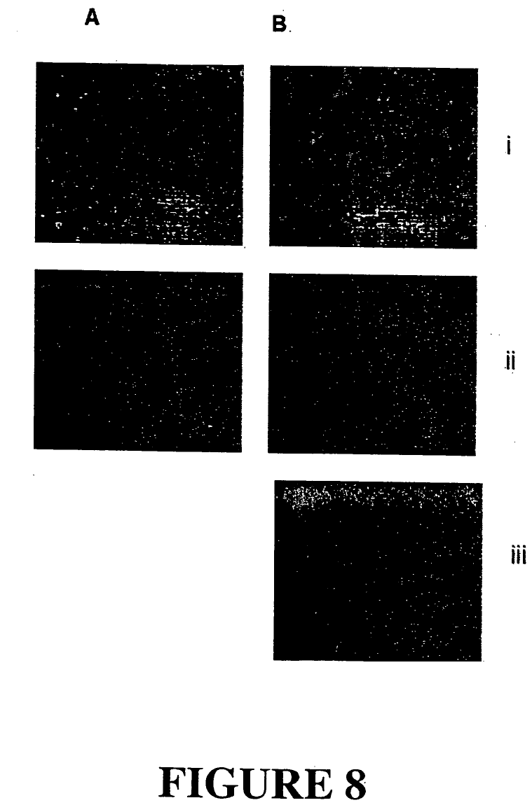

FIG. 8: Reconstructed ES cells

Cytoplasts were prepared from 4N ES cells, and fused with neurectoderm cells carrying an Oct4-GFP construct. Neurectoderm cells used for fusion did not express the Oct4-GFP construct prior to fusion.

Column A Control 4N ES cells.

Column B Reconstructed ES cells.

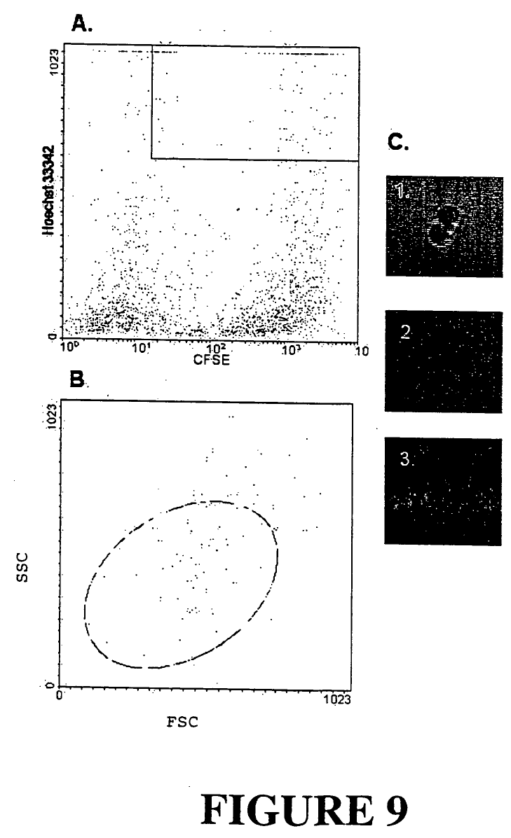

FIG. 9: Post-fusion enucleation

A: Multiple laser flow cytometry of large ES cells following post-fusion enucleation, with CFSE corresponding to the recipient cell and Hoechst 33342 to the nucleus of the donor cell B. Scatter plot of cells selected as positive for CFSE and Hoechst 33342 (gate indicated by inset rectangle in A.).

B: The ellipse indicates the area of the plot where 2N ES cells are normally found. Side Scatter (SSC) provides a measure of cell integrity. Front Scatter (FSC) provides a measure of cell size.

C: Epifluorescence microscopy of two positive cells with single nuclei after FAC sorting and restaining with H33342.

1 Phase contrast

2 The same field under UV2A fluorescence. Note staining with Hoechst 33342 (nucleus staining).

3 Same field under fluorescence microscopy (FITC Filter). Note staining with CFSE (protein staining).

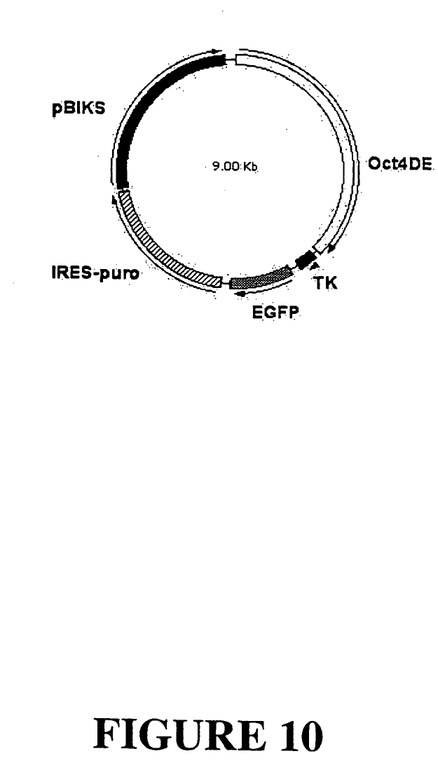

FIG. 10: Plasmid map for the Oct4-TK-GFP construct pBI-Oct4DE-TK-EGFP-IRES-puro

DE-Oct4 distal enhancer, TK—tyrosine kinase promoter, IRES—Internal Ribosome Entry Site.

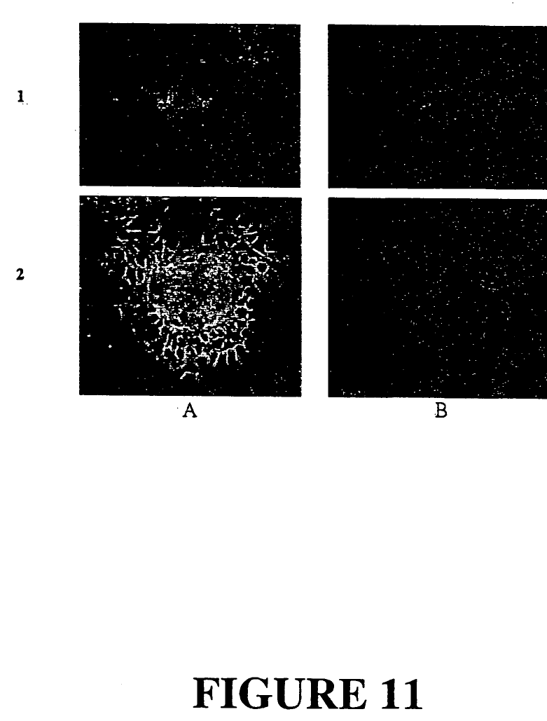

FIG. 11: GFP expression pattern of Oct4-TK-GFP cells

Expression patterns for (1) ES 2.5 cells transformed with a cytoplasmic GFP construct expressed in pluripotent and differentiated cells. GFP expression is visible in differentiated cells surrounding an ES cell colony and (2) Oct4-TK-GFP ES cells where GFP expression is developmentally regulated and only present in pluripotent cells. Note the complete disappearance of GFP expression in the differentiated cells surrounding the ES cell colony. (A: Phase contrast; B: same field under fluorescence microscopy (FITC filter); Magnification 20×.).

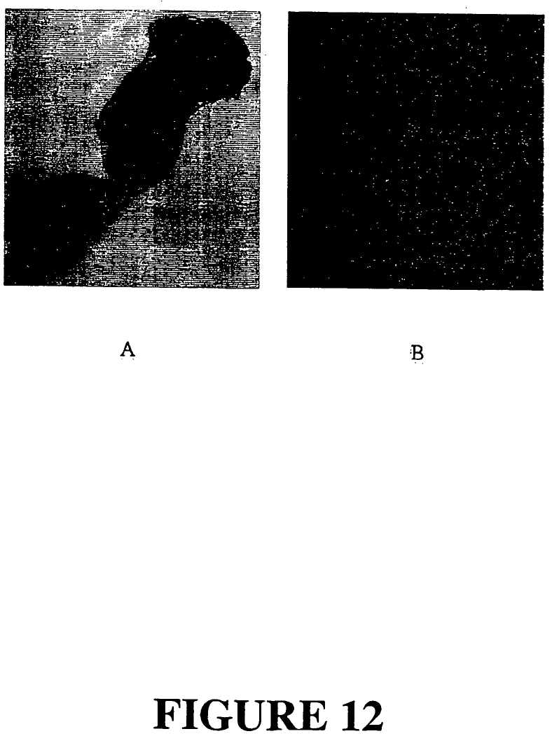

FIG. 12: Oct4-TK-GFP neurectoderm

EBM⁹ bodies with small pockets of GFP expressing cells. (A: Phase contrast; B: same field under fluorescence microscopy (FITC filter); Magnification 10×.).

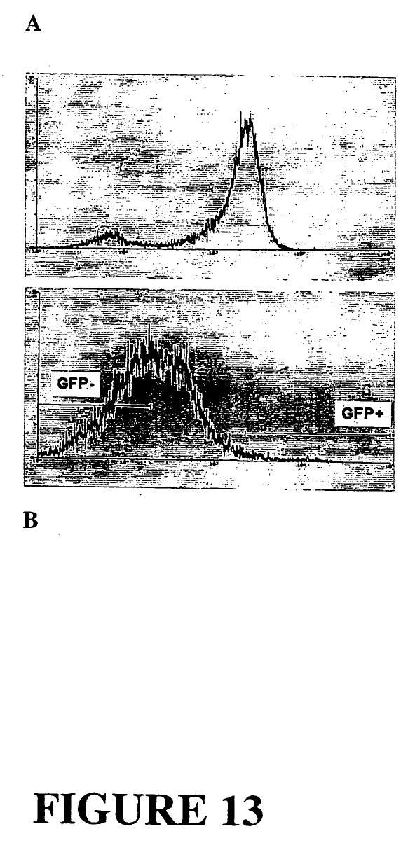

FIG. 13: FACS profile of neurectoderm cells

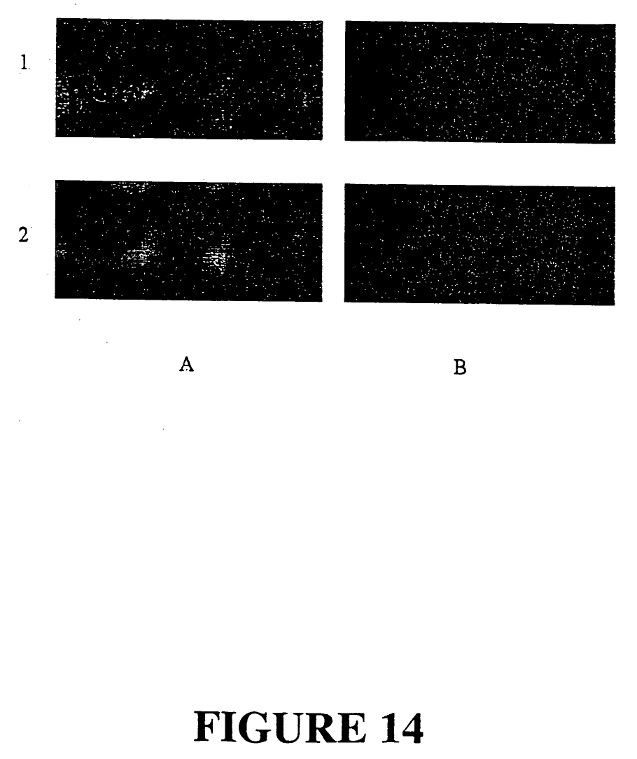

GFP FACS profile of Oct4-TK-GFP ES cells (A) and disaggregated EBM⁹ (B). For EBM⁹ cells the GFP profile is markedly shifted towards the left. Cells defined by the gate ìGFP-ì were sorted out and the absence of GPF expression was confirmed by epifluorescence microscopy (see FIG. 14).

FIG. 14: FACS analysis of neurectoderm

Disaggregated cells from EBM⁹ produced from Oct4-TK-GFP cells, (1) prior to FAC sorting, the presence of a few GFP expressing cells can be observed, and (2) after FAC sorting for GFP negative cells, none of the cells present express GFP. (A: Phase contrast; B: same field under fluorescence microscopy (FITC filter); Magnification 20×.).

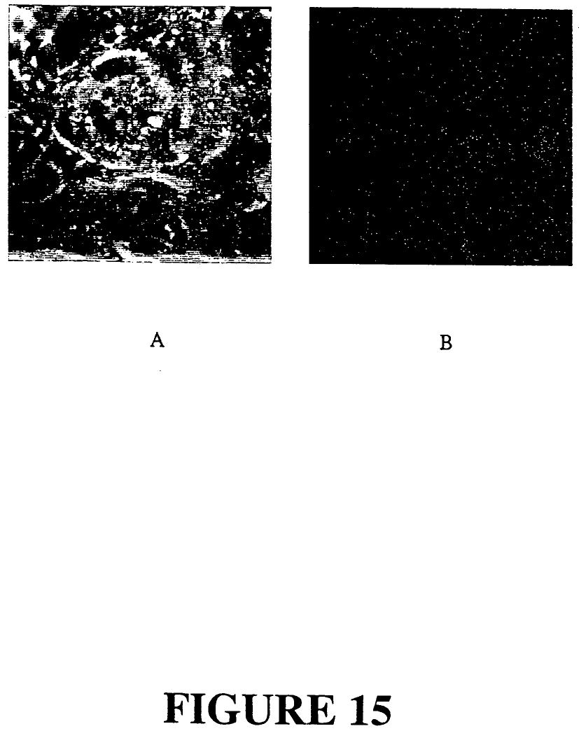

FIG. 15: Reprogramming of neurectoderm-reconstruction

Cytoplasts from tetraploid ES cells (RO.5) were fused with GFP negative Oct4-TK-GFP EBM⁹ cells. Day 3 post reconstruction, a group of cells is re-expressing GFP. (A: Phase contrast; B: same field under fluorescence microscopy (FITC filter); Magnification 20×.).

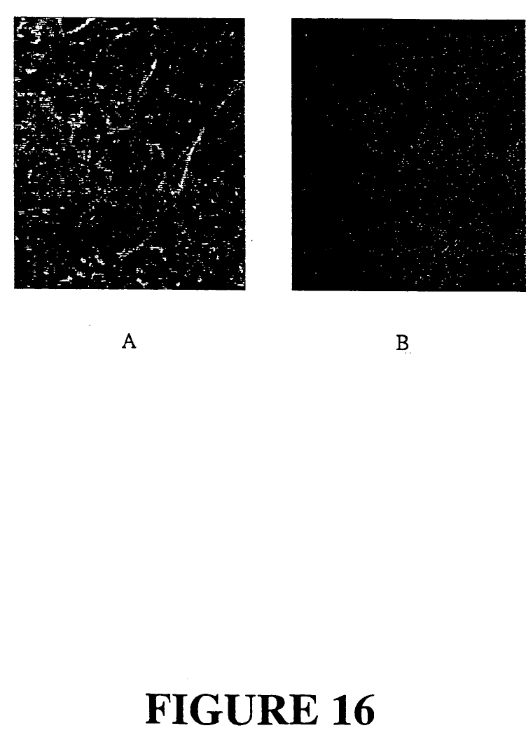

FIG. 16: Reprogramming of neurectoderm-fusion.

Intact ES-D3 cells were fused with GFP negative Oct4-TK-GFP EBM⁹ cells. Day 3 post fusion, a group of cells is re-expressing GFP. (A: Phase contrast; B: same field under fluorescence microscopy (FITC filter); Magnification 20×.).

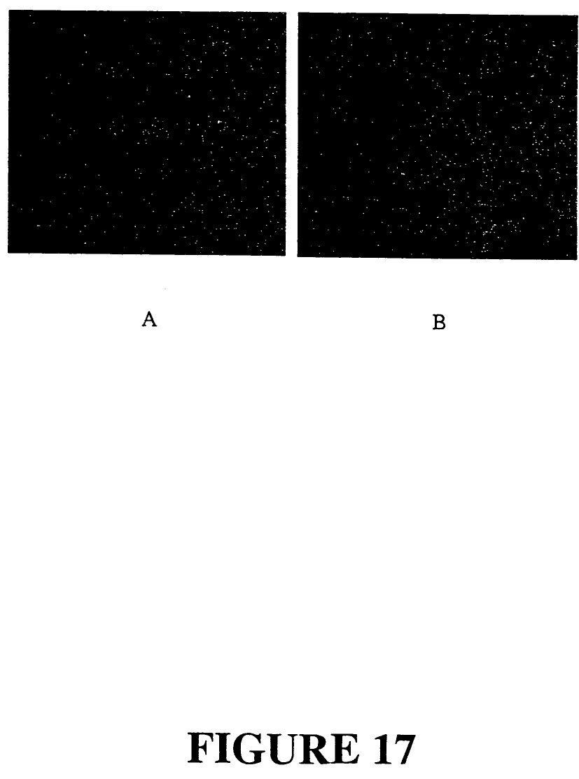

FIG. 17: Reprogramming of neurectoderm-morula aggregation.

GFP negative Oct4-TK-GFP EBM⁹ cells were micro-injected into the perivitelline space of compact morula stage murine embryos. Day 1 post microinjection, an early blastocyst stage embryo with a region of GFP expression was observed. (A: Phase contrast; B: same field under fluorescence microscopy (FITC filter); Magnification 20×.).

### EXAMPLES

### Example 1

### Fusion of ES Cells to form Large Cells as a Source of Cytoplasts

### 1. Fusion of ES Cells in Suspension by Electrical Pulse

Electrofusion conditions were optimised for the formation of large cells for cytoplast preparation.

### Method

ES cells were fused in 10 μl activation buffer (0.3 M mannitol, 100 μM CaCl₂, 100 μM MgSO₄, 0.01% polyvinylalcohol) using a BTX Electro Cell Manipulator ECM 2001. The suspended cells were placed in a fusion chamber (450-10WG, BTX, CA), between electrodes separated by a gap of 1 mm. Cells were exposed to two DC fusion pulses (60 μsec; 200 V, 250 V or 300 V), 5 sec apart, with or without pre and post AC pulses (5 V, 5 secs).

Multinucleate cells (cells containing two or more separate nuclei) were identified by fluorescent staining of DNA with Hoechst 33342.

### Results

TABLE 1

| Electrofusion Treatment | n | Multinucleate cells (%) | Lysed cells (%) |
|---|---|---|---|
| +AC; 200 | 50 | 10 (20%) | 6 (3%) |
| +AC; 250 | 60 | 13 (22%) | 27 (45%) |
| +AC; 300 | 32 | 0 (0%) | 28 (88%) |
| −AC; 300 | 58 | 6 (10%) | 26 (45%) |
| Control (unfused) | 79 | 1 (1%) | 0 |

+AC: An AC pulse of 5V for 5 secs was administered pre and post the delivery of the DC fusion pulse

### Conclusion

Electrofusion of ES cells can be achieved using all DC voltages tested from 200-300 Volts. Cell lysis occurred less frequently at 200V DC.

### 2. Fusion of ES Cells in Culture using Polyethylene Glycol (PEG)

This method mediates fusion between cells by exploiting the natural cell-cell contact between cells in monolayer culture.

### Method

ES cells were grown to near confluence in small petri dishes.

Cells were washed twice in Ca free PBS at 37° C.

PEG solution (50% PEG₆₀₀₀ in Hepes buffer at 37° C.) was added rapidly, and left from 30 sec to 3 min.

Cells were washed twice in Ca free PBS, fed with ES cell medium supplemented with LIF and foetal bovine serum, and incubated for 24 h.

Cells were trypsinised and viewed after staining with Hoechst 33342 to identify large, multinucleate cells.

### Results

TABLE 2
PEG fusion of attached ES cells

| | Cells counted | No. large/multi-nucleate cells (%) |
|---|---|---|
| PEG fused | 420 | 53 (12.6%) |
| Unfused | 300 | 7 (2.3%) |

The number of multinucleate cells derived from two experiments is shown in Table 2. Routinely in a number of additional experiments, fusion rates of 10-30% were observed following PEG fusion of attached ES cells in a monolayer.

The following cell types were observed after PEG treatment.

Large, binucleate cells.

Large cells with a single large nucleus.

Trinucleate cells.

Cells with visible metaphase.

Normal size ES cells

Large cells were seen at a significantly lower frequency in the absence of PEG treatment. The large cells have an estimated diameter of about 20 μm, as opposed to 15 μm for unfused cells. Cells with a diameter of 20 μm have a volume approximately double that of unfused cells.

FIG. 1 shows large fused cells in suspension, produced by PEG fusion (B), compared to smaller unfused ES cells (A).

### Conclusion

Cell fusion can be achieved by PEG treatment of ES cells in culture.

This process gives rise to cells larger, and containing more cytoplasm than diploid (2N) ES cells.

NB: alternatively, ES cells in suspension can also be fused with PEG.

### 3. Electrical Fusion of ES Cells in the Presence of PEG

The method of Li and Hui (1994), which describes the fusion of erythrocytes, was adapted to ES cell fusion.

### Method

10 μl of ES cells in suspension w re added to fusion medium (0.3 M mannitol)±10% PEG₈₀₀₀.

Cells were exposed to a DC fusion pulse (60 μsec; 300 V), with pre- and post-AC pulses (4 V, 5 secs).

Cells were transferred to Eppendorf tubes, and nuclei were stained with Hoechst 33342.

### Results

TABLE 3

| | | Single | Multinucleate | |
|---|---|---|---|---|
| Treatment | n | nuclei | cells (%) | Lysed cells (%) |
| Electrical pulse | 97 | 50 | 9 (9%) | 27 (28%) |
| PEG | 110 | 104 | 4 (4%) | 2 (2%) |
| PEG + Electrical | 106 | 36 | 21 (20%) | 49 (46%) |
| pulse | | | | |

### Conclusion

PEG in combination with electrofusion is an effective method for fusing (ES) cells.

### 4. Phytohaemagglutinin Treatment

Phytohaemagglutinin (PHA) agglutinates single cells, increasing the proportion of cells in cell-cell contact, and thereby potentially facilitating cell fusion.

### Method

Trypsinized cells were incubated for 5 min in cell fusion medium containing 200 μg/ml PHA. Cells were examined for agglutination and then subjected to electrofusion as described above.

### Results

Results are shown in FIG. 2. FIG. 2.A shows that PHA treatment promotes the formation of groups of cells in a single cells suspension. This in turn increases the efficiency of fusion for these cells (FIG. 2.B).

### Conclusion

PHA treatment promotes the agglutination of ES cells.

Agglutination through PHA treatment increases the efficiency of ES cells fusion. This method could be also used to promote the fusion of cytoplasts and karyoplasts (reconstruction).

### 5. Summary

Large multinucleate or polyploid cells can be formed from normal diploid ES cells, through cell fusion using PEG, electrofusion or a combination of both. The efficiency of this process can be improved by treatment with PHA.

### Example 2

### Production of a Line of Fused Cells

The aim of the study was to produce a line of polyploid/tetraploid cells to be used as a reprogramming vehicle for differentiated or partially differentiated cells.

### Methods

Two ES cell lines were fused, one of which was transfected with a construct conferring resistance to puromycin, while the other was transfected with a construct conferring resistance to neomycin.

Following PEG fusion, double antibiotic selection (puromycin and the neomycin analog G418) was applied for up to 10 days to select for fused cells. Individual resistant colonies were expanded and cryopreserved. Two lines were maintained in medium containing both antibiotics and analysed for expression of pluripotent markers.

1. F1 was derived from a mixture of colonies that survived double antibiotic selection, and

2. F2.1 was derived from a single surviving colony following double antibiotic selection.

### Results

TABLE 4

| | Control | F1 cells (mixed | F2.1 cells |
|---|---|---|---|
| | ES cells | colonies | (single colony) |
| Puro^{R}/Neo^{R} colonies | − | − | + |
| Large cells | − | + | + |
| Proliferation | + | + | + |
| Colony morphology (ES | + | +/− | + |
| cell like) | | | |
| Tetraploid karyotype (4N) | − | + | + |
| Alkaline phosphatase activity* | + | + | + |
| Oct4 expression* (in situ | + | + | + |
| hybridisation) | | | |
| ECMA7 staining* | + | + | + |
| (Immunohistochemistry) | | | |

*Alkaline phosphatase, Oct4 and ECMA7 are pluripotent cell markers.

FIG. 3 shows F2.1 cells (B), compared to smaller unfused ES cells (A).

### Conclusion

The method described above led to the production and isolation of tetraploid ES-like cells that retain pluripotent properties. The method is simple, robust and practical. Antibiotic selection is used to maintain the polyploid cells as a homogeneous population.

The tetraploid cells so formed are larger than normal diploid ES cells. The tetraploid cell line potentially provides a source of large cytoplasts, containing increased amounts of cytoplasmic factors for reprogramming. The tetraploid cell can also be used to form a heterokaryon/hybrid cell by fusion with a differentiated cell or karyoplast, leading to the subsequent reprogramming of the differentiated nucleus. When the pluripotent nucleus and differentiated nucleus remain separate, or unfused, the pluripotent nucleus may be later expelled by centrifugal, chemical or other means (see Example 6).

We have also produced a line of very large ES cells, where 4N ES cells produced as described above were fused with 2N ES cells carrying resistance to hygromycin B (data not shown). Fused cells were selected with a combined treatment of G418 and hygromycin B. These cells retained the expression of the pluripotent cell marker Oct4. These cells provide a further advantage over 4N cells in term of large cytoplasmic content for cell reprogramming.

Similarly 4N and 6N cells may be fused to each other to give cells/cell lines with a still larger karyotype and cell volume. These cells can be selected by fluorescence activated cell sorting (FACS) or any other means.

### Example 3

### Production of Cytoplasts by Enucleation of Fused ES Cells

### 1. Enucleation by Micromanipulation of ES Cells Fused by Electrical Pulse

### Method

Fused ES cells were manipulated with an enucleation pipette in the presence of a cytoskeletal inhibitor (cytochalasin B) to prepare cytoplasts. The enucleation pipette was constructed with a blunt tip with an internal diameter of 6-10 μm. Cytoplasm, bound by the cell membrane was gently aspirated into the pipette until the droplet (cytoplast) was pinched off from the cell. Cytoplasts can also be aspirated from unfused (normal) ES cells but they are generally smaller in size. Enucleation of cytoplasts is confirmed with Hoechst 33342 DNA staining and visualisation under UV2A light.

### Results

Cytoplasts were prepared by micromanipulation of fused pluripotent cells (data not shown).

### Cemments

The use of a small ID (˜6 μm) enucleation pipette prevented the accidental aspiration of the nucleus into the pipette.

Droplets of cytoplasm could be easily pinched off from fused cells.

Droplets were ˜⅓ the diameter of intact cells, indicating that cytoplasts of suitable size can be produced by further fusion of 2-4 cytoplasm droplets.

### 2. Enucleation of Large ES Cells using Percoll Density Gradients

### Method

The method by Tatham et al. (1995) was adapted to the enucleation of large ES cells. This method eliminates the need for micromanipulation of isolated ES or other cells and allows the bulk production of enucleated cells and karyoplasts. The method relies on differential organelle density, with nuclei being the heaviest organelle within cells. ES cells were first fused into large cells using an electrical pulse as described above, in order to increase the final size of cytoplasts. Cells were then treated with the cytoskeleton-disrupting agent cytochalasin B. Cells were then submitted to a first centrifugation at 15000 g for 2 min, resulting in the stratification of organelles within the cytoplasm and preparing the cells for enucleation. Cells were then loaded on the top of a discontinuous Percoll density gradient (7.5%, 30% and 45% with cytochalasin B), and centrifuged for 5 sec at 5000 g.

### Results

Cytoplasts and karyoplasts prepared using Percoll gradient centrifugation are shown in FIG. 4.

Extruded nuclei and karyoplasts were found enriched in the 45% layer, whereas cytoplasts were found in the 7.5% layer. Enucleation of cytoplasts was confirmed with Hoechst 33342 DNA staining. When cytoplasts were prepared from transfected ES cells expressing cytoplasmic GFP, they fluoresced under FITC but showed no Hoechst 33342 fluorescence, whereas the reverse was observed for karyoplasts.

Cytoplasts could be enriched up to 80% by diluting and recentrifuging the 7.5% layer for 1 min at 15000 g to .eliminate contaminating intact cells. An enriched cytoplast preparation is shown in FIG. 5.

### 3. Centrifugal Enucleation

### Method

The method by Celis and Celis (1994) was adapted to the enucleation of fused and unfused ES cells. The method involved seeding ES cells on gelatine or cellular fibronectin (cFN) coated plastic disks, and inverting the disks into a round bottom centrifuge tube containing media supplemented with the cytoskeletal inhibitor cytochalasin B. The tubes were subsequently centrifuged at high speed (˜15,000 g) for 20 to 45 min. The treatment resulted in extrusion of nuclei, with a minimum of surrounding cytoplasm, into the media (karyoplasts). Enucleated cytoplasts remained attached to the disks. Cytoplasts were distinguished from non-enucleated ES cells using Hoechst 33342 staining.

### Results

TABLE 5

| | Total cells | Nucleated | Cytoplasts |
|---|---|---|---|
| DISKS | counted | cells | (% of total) |
| Unfused cells | 79 | 70 | 9 (11) |
| Fused cells | 128 | 87 | 41 (32) |
| Fused cells* | 193 | 22 | 171 (89) |

*4N cell line +cFN coated disk

FIG. 6 shows cytoplasts and intact cells attached to the disk after centrifugal centrifugation. Note that larger cells produced larger cytoplasts.

### Conclusion

Centrifugal enucleation can be used to prepare cytoplasts from ES cells. The use of large cells reduced detachment of cytoplasts from the disks and strongly increased the cytoplast yield. This was further improved by the use of a homogenous polyploid (4N) cell line in conjunction with the use of cFN coated disks. Fused (large) cells resulted in larger cytoplasts, and therefore have advantages over normal diploid pluripotent cells in reprogramming.

### Example 4

### Production of Karyoplasts using Centrifugal Enucleation

This method involves seeding cells on a gelatinise or cFN coated plastic disk, inverting the disk into a round bottom centrifuge tube containing media supplemented with the cytoskeletal inhibitor cytochalasin B. The tubes are subsequently centrifuged at high speed (˜15,000 g) for 45 min. Karyoplasts are extruded from the cells with a minimum of surrounding cytoplasm and collected from the media. Indeed, karyoplasts prepared from ES cells expressing cytoplasmic GFP do not fluoresce under FITC but show Hoechst 33342 fluorescence. Cytoplasts remain attached to the discs (data not shown).

Contamination of the karyoplast preparation with intact cells that detach during centrifugation can be greatly reduced by plating the karyoplast preparation in gelatinised dishes for 2-4 hrs. This treatment results in preparations made up of greater than 99% of karyoplasts (n=3) in the supernatant as intact cells readily adhere to the gelatinised substrate whereas karyoplasts do not adhere to the substrate and remain in suspension.

### Example 5

### Reconstruction

### Method

Cytoplasts and karyoplasts produced by any of the above methods can be fused together using electrical fusion or PEG mediated fusion. Karyoplasts can also be fused to intact ES cells. The use of an agglutination treatment to increase cell to cell contact can optionally be used.

Reconstruction using PEG was carried out as follows:

Cytoplasts were prepared from polyploid ES cells using centrifugal enucleation. Use of larger cells facilitated the reconstruction process. Cytoplasts were left to recover from cytochalasin B treatment and enucleation by incubation in normal ES cell medium.

Karyoplasts were also prepared by the centrifugal method, and enriched by removal of contaminating intact cells by their adhesion onto gelatinized plates as described previously.

The enriched karyoplast suspension was recentrifuged onto cytoplasts or intact cells for 15 min at 14500 g at room temperature, in the presence or in the absence of 100 μg/ml phytohaemagglutinin.

Karyoplasts agglutinated to cytoplasts or intact cells were fused with 50% PEG₆₀₀₀ (in Hepes buffer pH 7.55) for 2 min at 37° C.

Reconstructed cells were selected on the basis of antibiotic resistance carried by the karyoplast nuclei. FAC sorting could be further used in order to separate diploid/tetraploid cells from unwanted cells of higher ploidy.

### Results

A) This protocol was first used to fuse ES cell karyoplasts to intact ES cells.

Intact ES cells that do not express GFP but carry resistance to neomycin, were used as recipient cells.

Karyoplasts were prepared from ES cells expressing cytoplasmic GFP, and carrying puromycin resistance. Expression of GFP was not detectable in these karyoplast as they were virtually devoid of cytoplasm. Karyoplasts were fused to intact cells GFP negative cells as described above, after centrifugation in the presence of phytohaemagglutinin (FIG. 7 (i) A). The resultant (hybrid/) fused cells did not express GFP immediately following fusion (FIG. 7 (i) B).

One day after fusion, expression of GFP could be detected in patches of these cells (FIG. 7, (ii). A and B). Following treatment with puromycin and the neomycin analog G418, cells that did not receive a nucleus from a karyoplast did not survive (FIG. 7, (iii) A and B). At the end of antibiotic treatment colonies of double-antibiotic resistant, GFP-expressing cells were obtained (FIG. 7, (iv) A and B).

B) The reconstruction protocol was also applied to the fusion of ES cell karyoplasts to cytoplasts prepared from 4N ES cells.

4N cells were grown and cytoplasts prepared using the centrifugal enucleation method. Prior to enucleation these cells were stained with Hoechst 33342. Karyoplasts were prepared as described in Example 4 and fused to cytoplasts using methods described for intact cells. FIG. 8 shows intact 4N cells (A) and reconstructed cells (B) 3 days after enucleation and reconstruction. FIG. 8 (i): phase contrast microscopy showed that reconstructed cells formed ES cell like colonies. (ii): an absence of Hoechst 33342 staining showed that reconstructed cells were devoid of nuclei from the original 4N cells. (iii): GFP expression could be observed in reconstructed cells.

Note that when 2N cells were used for the preparation of cytoplasts, no reconstruction was obtained.

### Conclusion

Reconstruction was achieved by fusion of karyoplasts to intact or large enucleated ES cells. Reconstructed cells were viable and expressed a transgene carried by the karyoplast nucleus.

### Example 6

### Post-Fusion Enucleation

This is an alternative method for the production of reconstructed cells, i.e. cells where the cytoplasm and plasma membrane originate from one cell type (A) and the nucleus originates from a different cell type (B). Typically, but not exclusively, cell A is a pluripotent cell, and cell B is a partially or terminally differentiated cell, and the purpose of the reconstruction step is to achieve the reprogramming of cell B. An intact pluripotent cell is fused with an intact differentiated cell or a karyoplast from such a cell, to form a heterokaryon. Fusion can be achieved by PEG or electrofusion, as described in Example 1. The pluripotent cell nucleus is then removed. When compared with the production of reconstructed cells though the fusion of cytoplasts with intact cells or karyoplasts (see Example 5), the present method has the advantage of preserving the integrity of the pluripotent cell before the critical fusion step is carried out. Also, the presence of the pluripotent cell nucleus for a time prior to removal ensure the continuous supply of factors important for the reprogramming of the differentiated nucleus. The critical step in this technique is the selective removal of the pluripotent nucleus from the heterokaryon. If a polyploid pluripotent cell is used as the recipient cell, centrifugal enucleation conditions can be applied which result in the selective removal of the large nucleus. Therefore, use of large, polyploid cells has significant advantages in heterokaryon use for reprogramming.

### Method

This example describes the application of the method outlined above to the fusion of 2N ES cells with 4N or 6N ES cells, the removal of the 4N/6N ES cell nucleus, and the identification of reconstructed cells. 4N and 6N cells obtained as described in Example 2 and grown on plastic disks were loaded with the protein dye CFSE. 2N ES cells were trypsinized and stained with the nuclear fluorescent dye Hoechst 33342. These cells were fused to the 4N and 6N cells using PEG, and the fused cells returned to the incubator overnight. At this stage, the cells can be treated with a cell cycle arrester in order to maintain the two nuclei separate, although it was found in this experiment that most cells do not undergo division soon after fusion. The fused cells were then submitted to centrifugal enucleation as described in Example 3, with a centrifugation time of 10 min. After recovery, cells were trypsinized and examined under fluorescence microscopy. Using multiple-laser flow cytometry, cells were then sorted for size and co-localization of CFSE and Hoechst 33342. Positive cells were replated on gelatin-coated dishes and restained with Hoechst 33342. Cells were re-examined under fluorescence microscopy for size, Hoechst 33342 staining, number of nuclei and CFSE staining. A photographic record was maintained.

### Results

FIG. 9(A) shows that following post-fusion enucleation a population of cells could be identified that were both positive for CFSE and Hoechst 33342. FIG. 9.(B) shows that these double-positive cells were found in a range of sizes, some of which corresponded in size with 2N ES cells. Epifluorescence microscopy confirmed the co-localization of Hoechst 33342 and CFSE in these cells. Finally, photographic evidence shown in FIG. 9(C) indicated the presence of a single nucleus in these cells, confirming the expulsion of the pluripotent nucleus.

### Conclusion

Post-fusion enucleation in hetokaryons resulting from the fusion of pluripotent large ES cells and normal ES cells was achieved using differential centrifugation conditions for selective expulsion of the heavier pluripotent nucleus.

When a differentiated or partially differentiated cell is reprogrammed in a heterokaryon constructed from a aneuploid, euploid or polyploid pluripotent cell, the heavier pluripotent nucleus may be removed by the method described here, resulting in the formation of a diploid reprogrammed cell.

### Example 7

### Production of a Line of ES Cells Expressing Cytoplasmic GFP Undereth Control of Oct4

The Oct4 gene is developmentally regulated and its expression is required for pluripotent cell maintenance. An ES cell line, which expresses cytoplasmic GFP under the control of an element of the Oct4 promoter, was generated by stable transfection. Differentiated cells, such as neurectoderm, produced from these cells are not pluripotent, and should not express Oct4 and therefore should be GFP negative. A return of GFP expression indicates that these neurectoderm cells are reprogrammed (i.e; reverted to a pluripotent state where Oct4 is expressed).

### Methods

A plasmid where expression of GFP and puromycin resistance are placed under the control of the tyrosine kinase (TK) promoter and the distal enhancer (DE) of the Oct4 gene (pBIOct4DE-TK-EGFP-IRES-puro) was constructed as shown in FIG. 10, and used to transfect ES D3 cells. Clones with stable transfection were selected with puromycin and cell lines were produced. One cell line was expanded and the ES cells were allowed to differentiate by withdrawing LIF.

### Results

The Oct4-TK-GFP cell line expressed cytoplasmic GFP in cells maintained as ES cells. Following LIF withdrawal a complete loss of GFP expression was observed in only those cells that differentiated (See FIG. 11). Puromycin selection resulted exclusively in survival of cells with an ES cell morphology. Therefore both GFP expression and puromycin selection can be employed to identify and isolate cells that have reprogrammed to a pluripotent state (as determined by Oct4 expression).

### Conclusion

An ES cell line that expresses GFP under the control of the Oct4 promoter, was generated. The use of these cells as a source of differentiated nuclei for reprogramming experiments, provides a rapid assay system to assess reprogramming, where the loss of GFP expression indicates loss of pluripotency, and a return of GFP expression indicates reprogramming.

### Note

An ES cell line was also generated, where the pBIOct4DE-TK-EGFP-IRES-puro plasmid was co-transfected with a PGK-Hygro plasmid. This plasmid confers resistance to hygromycin under the control of the constitutive phospho-glycerate kinase (PGK) promoter. These cells present GFP expression and puromycin resistance properties that are identical to that of the original Oct4-TK-GFP cell line. They also present the extra advantage of being selectable by hygromycin at all stages of differentiation.

### Example 8

### Fluorescence Activated Cell Sorting (FACS) of Differentiated Oct4-TK-GFP ES Cells to Select GFP Negative Neurectoderm Cells

### Method

EBM⁹, which are essentially a body of neurectoderm cells, were produced from Oct4-TK-GFP ES cells following protocols described in PCT/AU01/00030 “Cell Production”. The bodies were observed for GFP expression using FITC microscopy. Some bodies displayed small pockets of GFP expression, while others were GFP negative. All the bodies were disaggregated using EGTA following standard protocols. The disaggregated cells were subjected to FAC sorting and GFP positive and negative cells were separated to form two cell populations. The GFP negative and positive cell populations were checked by FITC microscopy for GFP expression and photographed. The GFP negative cells were subsequently used for reprogramming experiments.

### Results

FACS analysis of disaggregated EBM⁹ derived from Oct4-TK-GFP cells showed that these bodies are mainly composed of GFP negative cells (FIG. 12). However a small population of GFP positive cells is present, which was successfully removed by FAC Sorting (FIG. 13; FIG. 14). The absence of endogenous Oct4 in these cells was also confirmed by in situ hybridisation (data not shown).

### Conclusion

FAC Sorting of disaggregated neurectoderm cells derived from EBM⁹, was a practical method to separate GFP positive and negative cells. Following sorting these cells were morphologically intact.

### Example 9

### Reconstruction of Neurectoderm Karyoplasts with Cytoplasts Derived from Pluripotent Tetraploid Cells

This study was conducted to examine reprogramming of differentiated neurectoderm cells (karyoplasts), derived from Oct4-TK-GFP ES cells, when they were reconstructed with cytoplasts produced from tetraploid ES cells. Reprogramming would be identified in the first instance by a return of GFP expression in the reconstructed cell.

### Methods

Intact GFP negative neurectoderm cells were prepared from EBM⁹ bodies as described in Example 8, and fused with cytoplasts produced from tetraploid ES cells (pluripotent) using methods described in Example 5. Reconstructed cells were placed in ES cell culture conditions and observed using both phase contrast and FITC microscopy and a photographic record was maintained.

### Results

At Day 3 post reconstruction, the presence of groups of cells that expressed GFP was observed and noted. Photographs were taken of reconstructed cells both before and following visualisation of the GFP positive colonies (FIG. 15).

### Conclusion

FAC sorted neurectoderm cells, derived from Oct4-TK-GFP cells, are capable of being used for reconstruction with cytoplasts from tetraploid cells. The return of Oct4-GFP expression in these cells, suggests that these neurectoderm cells can be reprogrammed to a pluripotent state after exposure to cytoplasm from tetraploid ES cells.

### Example 10

### Fusion of Neurectoderm Karyoplasts with Intact ES (Pluripotent) Cells

Intact GFP negative neurectoderm cells were used as karyoplasts, and fused with intact ES cells to form cell hybrids. Reprogramming is identified in the first instance by a return of GFP expression in fused cells. The fused cells can either be used as a model to examine the reprogramming of the differentiated nucleus, or as a method to generate reprogrammed diploid cells if the pluripotent nucleus can be expelled (by centrifugal, chemical or other means).

### Methods

Oct4-TK-GFP ES cells were differentiated for 9 days to produce neurectoderm (EBM⁹). Intact GFP negative neurectoderm cells selected by FACS were fused with intact ES cells (ES_D3). Cells were placed in ES cell culture conditions and observed using both phase contrast and FITC microscopy and a photograph record was maintained.

### Results

At Day 3 post fusion the presence of a number of groups of cells that expressed GFP was observed and noted (FIG. 16). Photographs were taken of fused cells both before and following visualisation of the GFP positive colonies.

### Conclusion

Neurectoderm cells, derived from Oct4-TK-GFP ES cells, are capable of fusion with ES cells, following. FACS analysis. On exposure to the nucleus and cytoplasm of intact ES cells, the return of GFP expression, which is under the control of Oct4 in these cells, strongly suggests that these neurectoderm cells are reprogrammed to a pluripotent state.

### Example 11

### Dedifferentiation of Neurectoderm Following Injection into Mouse Embryos

Oct4-TK-GFP neurectoderm cells were injected into murine embryos at the morula stage. Return of GFP expression provides a rapid indication of reprogramming.

### Methods

Oct4-TK-GFP ES cells were differentiated for 9 days to produce neurectoderm (EBM⁹). Between 10 and 20 intact neurectoderm cells, selected by FACS to be GFP negative, were injected into the peri-vitelline space (between the compact morula and the surrounding zona pellucida) of compact morula stage murine embryos. Embryos were cultured in vitro and observed using both phase contrast and FITC microscopy. A photographic record was maintained.

### Results

At Day 1 post injection, 2 of 17 embryos injected with neurectoderm cells showed regions of GFP expression. In one embryo, which had developed to the early blastocyst stage, the GFP expression was present as a “clump” of fluorescence within the embryo itself (FIG. 17), whereas in the second embryo the cells expressing GFP were contained within the peri-vitelline space and had not been incorporated into the embryo.

### Conclusion

Neurectoderm cells can be used for morula aggregation, following FACS analysis. On being injected into the peri-vitelline space of morula stage mouse embryos, the return of GFP expression, (under the control of Oct4 in these cells), strongly suggests that these neurectoderm cells were reprogrammed to a pluripotent state.

Whether the reprogramming is a result of cell-cell interactions between the neurectoderm and embryonic cells or due to extra-cellular factors being secreted by embryonic cells, or both, is not elucidated by this experiment.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

### References

1 Bjornson C R R, Rietze R L, Reynolds B A, Magli M C & Vescovi A L (1999). Science 283, 534.

2 Campbell K H, McWhir J, Ritchie W A and Wilmut I (1996). Nature 380, 64.

3 Celis J E and Celis A (1994). In: Cell Biology: A Laboratory Handbook. Academic Press.

4 Eglitis M A & Mezey E (1997). Proc Natl Acad Sci USA 94, 4080.

5 Ferrari G, Cusella-De Angelis G, Coletta M, Paolucci E, Stornaiuolo A, Cossu G and Mavilio F. (1998). Science 279,1528.

6 Gurdon J, The control of gene expression in animal development (Oxford University Press 1974)

7 Kikyo N and Wolffe A (2000). J Cell Sci 113, 555.

8 Li L H and Hui S W (1994). Biophys J 67, 2361

9 Matveeva N M, Shilov A G, Kaftanovskaya E M, Maximovsky L P, Zhelezova A I, Golubitsa A N, Bayborodin S I, Fokina M M and Serov O L. (1998). Mol Reprod Dev 50, 128

10 Munsie M J, Michalska A E, O'Brien C M, Trounson A O, Pera M F and Mountford P S. (2000). Curr Biol 10, 989.

11 Petersen B E, Bowen W C, Patrene K D, Mars W M, Sullivan A K, Murase N, Boggs S S, Greenberger J S and Goff J P. (1999). Science 284,1168.

12 Poste G (1972). Exp. Cell Res. 73, 273

13 Prather R S et al, (1987). Nuclear transplantation in the bovine embryo; assessment of donor nuclei and recipient oocyte. Biol Reprod. 37, 859-866.

14 Prescott D M, Myerson D and Wallace J (1971). Exp. Cell Res. 71, 480

15 Roos D S (1991) In: Membrane Fusion, ed. Wilschut and Hoekstra

16 Smith L C and Wilmut I (1989). Biol Reprod 40,1027.

17 Stice S L and RobI J M. (1988). Nuclear reprogramming in nuclear transplant rabbit embryos. Biol Reprod 39, 657-664.

18 Tada M, Tada T, Lefebvre L, Barton S C and Surani M A (1997). EMBO J 16, 6510.

19 Tatham B G, Dowsing A D, and Trounson A O (1995). Biology of Reproduction 53, 1088-1094.

20 Willasden S M (1986). Nuclear transplantation in sheep embryos. Nature 320, 63-65.

21 Wilmut I, Schnieke A E, McWhir J, Kind A J and Campbell K H (1997). Nature 385, 810.

1. A method for reprogramming differentiated or partially differentiated cells to a less differentiated or dedifferentiated state, which method includes
providing
a karyoplast of a differentiated or partially differentiated cell; and
a source of multinucleate, aneuploid, euploid or polyploid multipotent or pluripotent cells, or morula or early embryos, or derivatives thereof; and
placing the karyoplast in intimate contact with the multipotent or pluripotent cells, or morula or early embryos or derivatives thereof under conditions sufficient to permit at least partial reprogramming of the cells.

2. A method according to claim 1, wherein the differentiated or partially differentiated cells are autologous.

3. A method according to claim 2, wherein the differentiated or partially differentiated cells are selected from the group consisting of blood (including bone marrow), skin, muscle, adipocyte or neural cells.

4. A method according to claim 3, wherein the multipotent or pluripotent cell source is derived from cells selected from the group consisting of embryonic stem cells (ES cells), early primitive ectoderm-like cells (EPL cells), primordial germ cells (PG cells) and embryonic carcinoma cells (EC cells); or derivatives thereof or mixtures thereof.

5. A method according to claim 4, wherein the multipotent or pluripotent cell source is derived from embryoid bodies derived from ES or EPL cells in vitro.

6. A method according to claim 5, wherein the karyoplast is incorporated within the embryoid body or aggregated with cells of the embryoid body.

7. A method according to claim 1 wherein the reprogramming is conducted utilising a cytoplast derived from the polyploid, aneuploid or multinucleate multipotent or pluripotent cell source.

8. A method according to claim 7, wherein the multipotent or pluripotent cell source includes cytoplasts derived from ES, EPL, PG or EC cells.

9. A method according to claim 1, wherein, when multipotent or pluripotent cells are used, the cell contact step further includes
subjecting the cells to a cell fusion step.

10. A method according to claim 9, wherein the cell fusion step includes subjecting the cells to an electrical pulse or exposure to polyethylene glycol (PEG) or a combination thereof.

11. A method according to claim 1, wherein the multipotent or pluripotent cell source is a multinucleate or polyploid pluripotent cell.

12. A method according to claim 11, wherein the multinucleate or polyploid pluripotent cell is a polyploid ES or EPL cell; and
the differentiated or partially differentiated cells are blood or neural cells.

13. A method according to claim 1 wherein, when the cells are partially reprogrammed,
isolating the partially reprogrammed cells; and
repeating the contact step until reprogramming is complete.

14. A method for reprogramming differentiated or partially differentiated cells to a less differentiated state, or de-differentiated state, which method includes
providing
a karyoplast of a differentiated or partially differentiated cell;
a source of mammalian morula or early embryos or derivatives thereof; and
placing the karyoplast in intimate contact with morula or early embryos or derivatives thereof for a period sufficient to permit at least partial reprogramming of the cells.

15. A method according to claim 14 wherein a mammalian karyoplast is placed in the perivitelline space of an early embryo.

16. A method according to claim 14, which method further includes separating the reprogrammed cells from other cells.

17. A method according to claim 16, which method includes
providing a source of differentiated or partially differentiated cells genetically modified to include a selectable marker; and
selecting the reprogrammed cells utilising the selectable marker.

18. A method according to claim 16, wherein the selectable marker is a marker expressed in pluripotent or multipotent cells.

19. A method according to claim 18, wherein the selectable marker contains a gene construct encoding Green Fluorescent Protein (GFP) linked to an Oct 4 promoter.

20. A method according to claim 14, wherein the differentiated or partially differentiated cells are blood (including bone marrow), skin, muscle, adipocyte or neural cells.

21. A method according to claim 14 wherein, when the cells are partially reprogrammed,
isolating the partially reprogrammed cells; and
repeating the contact step until reprogramming is complete.

22. A method for reprogramming differentiated or partially differentiated cells to a less differentiated, or dedifferential state, by formation of a cell hybrid, which method includes
providing:
a source of karyoplasts of differentiated or partially differentiated cells;
a source of multipotent or pluripotent cells, morula or early embryos or derivatives thereof;
placing the karyoplasts in intimate contact with the multipotent or pluripotent cells;
subjecting the cells to a cell fusion step to form a cell hybrid;
subsequently removing from said reprogrammed cells, the multipotent or pluripotent cell nuclei; and
culturing the reprogrammed cells to permit proliferation thereof.

23. A method according to claim 22, wherein the differentiated or partially differentiated cell source is a somatic cell.

24. A method according to claim 23, wherein the differentiated or partially differentiated cells are selected from the group consisting of blood (including bone marrow), skin, muscle, adipocyte or neural cells.

25. A method according to claim 22, wherein the multipotent or pluripotent cells are multinucleate, aneuploid, euploid or polypoid multipotent or pluripotent cells.

26. A method according to claim 22 wherein the cell fusion step includes subjecting the cells to an electrical pulse or exposure to polyethylene glycol (PEG) or a combination thereof.

27. A method according to claim 22, wherein the cell fusion step is such that the nuclei of the cell components remain separated; and
subsequently removing from said reprogrammed cells, the multipotent or pluripotent cell nuclei.

28. A method according to claim 22 wherein
the somatic cell nucleus and the nucleus of multipotent or pluripotent cells are maintained as separate nuclei by maintaining the cells at low temperature and/or by utilising a cell cycle arrester, and/or a cytoskeletal inhibitor.

29. A method according to claim 22 wherein the multipotent or pluripotent cell nucleus is removed by differential centrifugation.

30. A method according to claim 22, wherein the reprogrammed cells exhibit spontaneous removal of the multipotent or pluripotent cell nuclei.

31. A method for reprogramming differentiated or partially differentiated cells to a less differentiated or dedifferentiated state, which method includes
providing
a source of cytoplasm from a multinucleate, aneuploid, euploid or polypoid multipotent or pluripotent cell;
a karyoplast derived from a partially or terminally differentiated cell differing from the cytoplasm source;
a suitable culture medium
combining the cytoplasm source and karyoplast to form a reconstructed cell; and
maintaining the reconstructed cell in the culture medium for a time sufficient to permit at least partial reprogramming of the karyoplast in the combined cell.

32. A method according to claim 31, wherein the source of cytoplasm is cytoplasm removed from ES or EPL cells or derivatives thereof, or cytoplasm derived by at least partial enucleation of said cells.

33. A method according to claim 31, wherein the karyoplast is derived from blood (including bone marrow), skin, muscle, adipocyte or neural cells.

34. A method according to claim 31, wherein the cell reconstruction step includes fusion of the karyoplast and cytoplast so that the cytoplast content interacts with the karyoplast and induces at least partial reprogramming of the nuclear material.

35. A method according to claim 34, wherein the fusion step includes subjecting the cell components to an electrical pulse or exposure to polyethylene glycol (PEG) or a combination thereof.

36. A method according to claim 31, wherein, when the cells are partially reprogrammed,
isolating the partially reprogrammed cells; and
repeating the reconstruction and maintenance steps until reprogramming is complete.

37. A method according to claim 31, wherein the multinucleate or polyploid pluripotent cell is a polyploid ES or EPL cell; and
the differentiated or partially differtiated cells are blood or neural cells.

38. A reprogrammed or partially reprogrammed cell, when produced according to any one of the preceding claims.

39. A method for preparing a cytoplast, which method includes
providing a multinucleate, aneuploid, euploid or polyploid multipotent or pluripotent cell;
subjecting the multipotent or pluripotent cell to an enucleation step; and
harvesting the cytoplast so formed.

40. A method according to claim 39, where the cytoplast is obtained by micromanipulation, density gravity centrifugation or centrifugal enucleation optionally in the presence of cytochalasin B.

41. A method for preparing a karyoplast which method includes
providing a partially or terminally differentiated cell;
subjecting the partially or terminally differentiated cell to an enucleation step; and
harvesting the karyoplast so formed.

42. A method according to claim 41, wherein the partially or terminally differentiated cell is a partially or terminally differentiated somatic cell.

43. A method according to claim 42, wherein the somatic cell is selected from the group consisting of blood (including bone marrow), skin, muscle, adipocyte or neural cell.

44. A method according to claim 41, wherein the karyoplast is obtained by micromanipulation, density gravity centrifugation or centrifugal enucleation, optionally in the presence of cytochalasin B.

45. A method for preparing a multinucleate, aneuploid, euploid or polyploid multipotent or pluripotent cell, which method includes
providing a source of multipotent and/or pluripotent cells; and
subjecting the cells to a fusion step to produce a multinucleate, aneuploid, euploid or polyploid cell.

46. A method according to claim 45, wherein the multipotent or pluripotent cell source includes two or more diploid cells.

47. A method according to claim 45, wherein the cell fusion step leads to the production of large multinucleate, aneuploid, euploid or polyploid cells that contain an increased pool of cytoplasm.

48. A method according to claim 45, wherein the pluripotent cells are embryonic stem (ES) cells or early primitive ectoderm-like (EPL) cells.

49. A method according to claim 45, wherein the cell fusion step includes fusion utilising electrofusion or PEG-modified fusion, or a combination thereof.

50. A method according to claim 45, wherein the cell so formed is a multinucleate or polyploid pluripotent cell.

51. A multinucleate, aneuploid, euploid or polyploid multipotent or pluripotent cell derived from fusion of two or more diploid cells.

52. A multinucleate, aneuploid, euploid or polyploid multipotent or pluripotent cell according to claim 51, wherein the cell is a multinucleate or polyploid pluripotent cell.

53. A multinucleate, aneuploid, euploid or polyploid multipotent or pluripotent cell capable of expressing at least one multipotent or pluripotent marker.

54. A multinucleate, aneuploid, euploid or polyploid multipotent or pluripotent cell according to claim 53, wherein the multipotent or pluripotent marker is Oct4 or alkaline phosphatase.

55. A multinucleate, aneuploid; euploid or polyploid multipotent or pluripotent cell according to claim 51, wherein the cell is derived from ES, EPL, PG or EC cells.

56. A cytoplast derived from a multinucleate, aneuploid, euploid of polyploid multipotent or pluripotent cell according to claim 51.

57. A cell line formed from the multinucleate, aneuploid, euploid or polyploid multipotent or pluripotent cell according to claim 51.

58. A cell line according to claim 57, which is stable in culture in vitro.

59. A method for identifying dedifferentiation of differentiated or partially differentiated cells, which method includes
providing a cell mixture including reprogrammed or partially reprogrammed cells, produced according to any one of claims 1 to 37
the reprogrammed cells being optionally modified to include a pluripotent marker; and
screening the cell mixture for the presence or absence of pluripotent activity.

60. A method according to claim 59, wherein, when the reprogrammed cells are modified to include a pluripotent marker,
subsequently screening the cell mixture for the presence or absence of pluripotent marker activity.

61. A method according to claim 59, wherein the reprogramming step includes
placing karyoplasts of differentiated or partially differentiated cells in intimate contact with multipotent or pluripotent cells or morula or early embryos under conditions sufficient to permit at least partial reprogramming of the cells.

62. A method according to claim 61, wherein the multipotent or pluripotent cells are multinucleate, aneuploid, euploid or polyploid multipotent or pluripotent cells derived from fusion of two or more diploid cells.

63. A method according to claim 62, wherein the reprogrammed cell construct includes an ES or EPL cell line genetically modified to express a marker gene.

64. A method according to claim 63, wherein the cell line is an ES cell line transfected with an Oct 4 TK-GFP construct from which a karyoplast is derived.

65. A method for the production of differentiated or partially differentiated cells, which method includes:
providing
a source of reprogrammed multipotent or pluripotent cells according to claim 38;
a suitable medium; and
culturing the reprogrammed cells, optionally in the presence of a selected growth factor, for a period sufficient to permit partial or terminal differentiation.

66. A method according to claim 65, wherein the reprogrammed cells are derived from autologous cells.

67. A differentiated or partially differentiated cell when produced according to the method according to claim 65.

68. A method for the production of a chimeric animal, which method includes animal, which method includes
providing
a source of reprogrammed multipotent or pluripotent cells according to claim 38;
a pregastrulation embryo;
introducing the multipotent or pluripotent cells into the pregastrulation embryo; and
monitoring chimera forming ability.

69. Use of reprogrammed cells according to claim 38, or their differentiated or partially differentiated progeny, for use in human cell therapy or transgenic animal production.

70. Use of reprogrammed cells according to claim 38, or their differentiated or partially differentiated progeny, for use in human or animal gene therapy.
